(19) United States
(12) Patent Application Publication
(10) Pub. No.: US 2012/0157838 A1
(43) Pub. Date: Jun. 21, 2012

(54) ULTRASOUND MONITORING OF AESTHETIC TREATMENTS

(76) Inventors: Yossef Ori Adanny, Mitzpe llan (IL); Avner Rosenberg, Bet Shearim (IL); Edward Kantorovich, Rehovot (IL)

(21) Appl. No.: 13/393,169

(22) PCT Filed: Sep. 15, 2010
(86) PCT No.: PCT/IL10/00751

§ 371 Date: Feb. 28, 2012

(30) Foreign Application Priority Data
Oct. 6, 2009 (US) 61248997

(51) Int. Cl. A61B 8/00

(52) U.S. Cl. 600/438

(57) ABSTRACT

The current method and apparatus employs ultrasound beams to precisely monitor in real time the temperature of a specific segment of tissue being treated. Additionally, the current method and apparatus also provides ultrasound thermo-control of aesthetic skin treatment sessions. Such sessions may include one or more aesthetic skin tissue treatments such as sub-dermal fat cells breakdown, lessening of the amount of sub-dermal fat, tightening of loose skin, tightening and firming of body surfaces, reduction of wrinkles in the skin and collagen remodeling.

### CROSS-REFERENCE TO RELATED APPLICATIONS

This application is being filed under 35 U.S.C. 371 and claims the benefit of the filing date of United States provisional application for patent that was filed on Oct. 6, 2009 and assigned Ser. No. 61/248,997 by being a national stage filing of International Application Number PCT/IL2010/000751 filed on Sep. 15, 2010, each of which are incorporated herein by reference in their entirety.

### TECHNICAL FIELD

The method and apparatus relate to the field of aesthetic body treatments and more specifically to a method and apparatus for precise real time ultrasound monitoring of aesthetic treatments applied to skin.

### BACKGROUND

Aesthetic treatment devices are operative to effect treatment to delicate skin tissue layers employing numerous therapies including thermotherapy consisting of the application of energy into the tissue in a form of light, RF, ultrasound, electrolipophoresis, iontophoresis and microwaves and any combination thereof.

All currently known methods used in the art raise a subject's skin temperature above normal to about 50-60 degrees Celsius at which tissue damage may occur. It is therefore imperative to be able to precisely monitor in real time the temperature of a specific segment of tissue being treated and use the acquired information to alter the course of treatment and maintain subject's safety.

In order to continuously monitor skin temperature, suitable sensors such as thermocouples or thermistors are commonly incorporated into electrodes or transducers through which the energy is applied to the skin. These sensors have limited ability to precisely monitor the effect of the treatment on the tissue being treated. The accuracy of their reading in real time as well as the dependability on the information they provide as to tissue temperature at a specific treatment area are limited.

The use of the aforementioned techniques of temperature monitoring does not obviate certain potential skin damage risk since, for example, the sensor response time depends on various parameters such as heat conductivity from the skin to the sensor and heat conductivity inside the sensor. This may result in possible skin damage before the sensor reduces or cuts off the heating energy applied to the skin. To some extent, this risk can be avoided by reducing the cut-off temperature operating limit for the sources of heating energy supplying energy such as optical radiation, RF energy, and ultrasound energy. However, this would limit the heating energy transmitted to the skin and the treatment efficacy.

Currently used methods employing ultrasound to determine tissue temperature changes are based on ultrasound echo reflection and ultrasound deflection and are highly influenced by attenuation and diffusivity rendering them highly inaccurate.

There is therefore a need for precise real-time monitoring and control of skin tissue temperature during skin tissue heating treatments using RF, Laser or any other form of heating energy. The use of ultrasound thermometry and thermocontrol as described in the current method and apparatus provides a precise, non-invasive solution for such a need.

### SUMMARY

The current method and apparatus employs ultrasound beams to precisely monitor in real time the temperature of a specific segment of tissue being treated. Additionally, the current method and apparatus also provides ultrasound thermo-control of aesthetic skin treatment sessions. Such sessions may include one or more aesthetic skin tissue treatments such as sub-dermal fat cells breakdown, lessening of the amount of sub-dermal fat, tightening of loose skin, tightening and firming of body surfaces, reduction of wrinkles in the skin and collagen remodeling.

In accordance with an exemplary embodiment of the current method and apparatus an applicator includes a housing including an ultrasound transmitter and receiver. The transmitter and receiver consist of one or more piezoelectric elements arranged in one or more spatial configurations. The transmitter emits ultrasound beams in pulse form into tissue at a Brewster's angle of incidence. The ultrasound beam pulses travelled through the tissue, generally parallel to the surface of the skin and/or along an inter-layer border between treated tissue layers, are emitted thereby at a Brewster's angle of incidence and received by the receiver. The receiver piezoelectric elements convert the received beam signals to electric pulses, which are then communicated to an apparatus controller.

In another exemplary embodiment of the current method and apparatus, the housing includes one or more pairs of transceivers each consisting of a first transceiver operative to emit ultrasound beams into the tissue at a Brewster angle of incidence and a second transceiver operative to receive ultrasound beams emitted by the first transceiver, propagated through the skin substantially in parallel to the surface thereof and emitted thereby at a Brewster's angle of incidence.

In yet another exemplary embodiment of the current method and apparatus, the controller is operative, in real time, to analyze the ultrasound beams for information regarding changes in propagation speed of the beams, which are indicative of temperature changes in the tissue through which the beams have travelled. The controller is also operative to compare the temperature changes to tissue treatment temperature range limits defined in a predetermined treatment protocol and determine the criticality of the changes in light of these defined range limits. The criticality may be determined, for example, by setting upper and lower temperature limits for treatment heating energy levels applied to skin during a specific treatment session. These limits may be further broken down into temperature ranges and categorized as to levels of criticality.

In accordance with still another exemplary embodiment of the current method and apparatus the controller is also operative to take one or more actions based on the temperature changes and criticality thereof. For example, the controller may be provided with a treatment protocol, defining action to be taken at each level of criticality. Such actions may include changing the course of treatment by, for example, increasing or decreasing the level of treatment heating energy application, changing the duration of treatment heating energy application or stopping the treatment session altogether, recording changes and criticality thereof in a database, displaying the information on a display, printing the information on a printout or alerting a user.

In accordance with yet another exemplary embodiment of the current method and apparatus the controller communicates the new determined tissue treating temperature to a power generator operative to excite oscillation of the transmitter piezoelectric elements.

In accordance with still another exemplary embodiment of the current method and apparatus the applicator also employs one or more sources of heating energy in a form of at least one of a group consisting of light, RF, ultrasound, electrolipophoresis, iontophoresis and microwaves.

It will be appreciated that the current method and apparatus may be employed during one or more aesthetic skin tissue treatment selected from a group consisting of sub-dermal fat cells breakdown, lessening of the amount of sub-dermal fat, tightening of loose skin, tightening and firming of body surfaces, reduction of wrinkles in the skin and collagen remodeling.

### BRIEF DESCRIPTION OF THE DRAWINGS

The present method and apparatus will be understood and appreciated from the following detailed description, taken in conjunction with the drawings in which:

FIG. 1A and FIG. 1B are simplified views of an exemplary embodiment of the current method and apparatus for precise ultrasound monitoring of treated skin temperature in real time employing a Brewster's angle of incidence.

FIG. 1C is a plan view of aesthetic treatment device applicator of FIG. 1A.

FIG. 2A is a simplified view of yet another exemplary embodiment of the current method and apparatus for ultrasound monitoring treated skin temperature in real time employing a Brewster's angle of incidence.

FIG. 2B is a plan view aesthetic treatment device applicator of FIG. 2A.

### GLOSSARY

The terms “transmitter”, “transceiver” and “receiver” used in the present disclosure mean devices that use piezoelectric elements to emit and/or receive ultrasound beams and may be used interchangeably, their functionality defined by their predetermined location in the apparatus and electric connection to a controller as will be described in detail hereinbelow.

The term “skin” and “skin tissue” may be used interchangeably in the present disclosure and mean the tissue layer consisting of epidermis, dermis and including dermal structures such as sebaceous glands, hair follicle, hair shafts, sweat glands, etc.

### DETAILED DESCRIPTION OF EXEMPLARY EMBODIMENTS

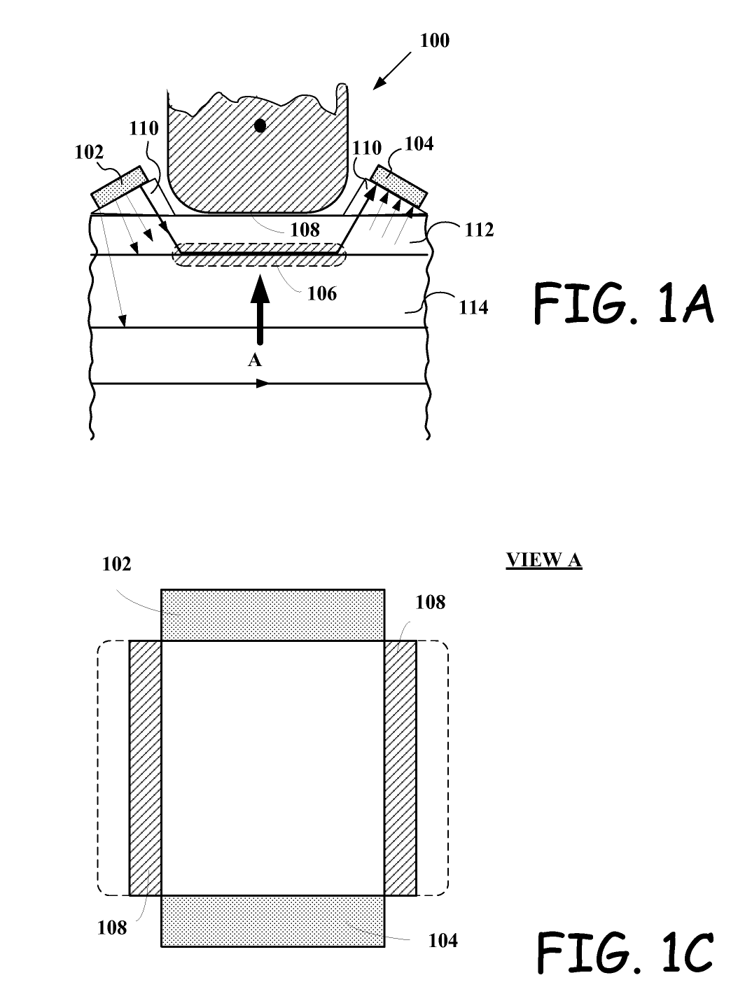

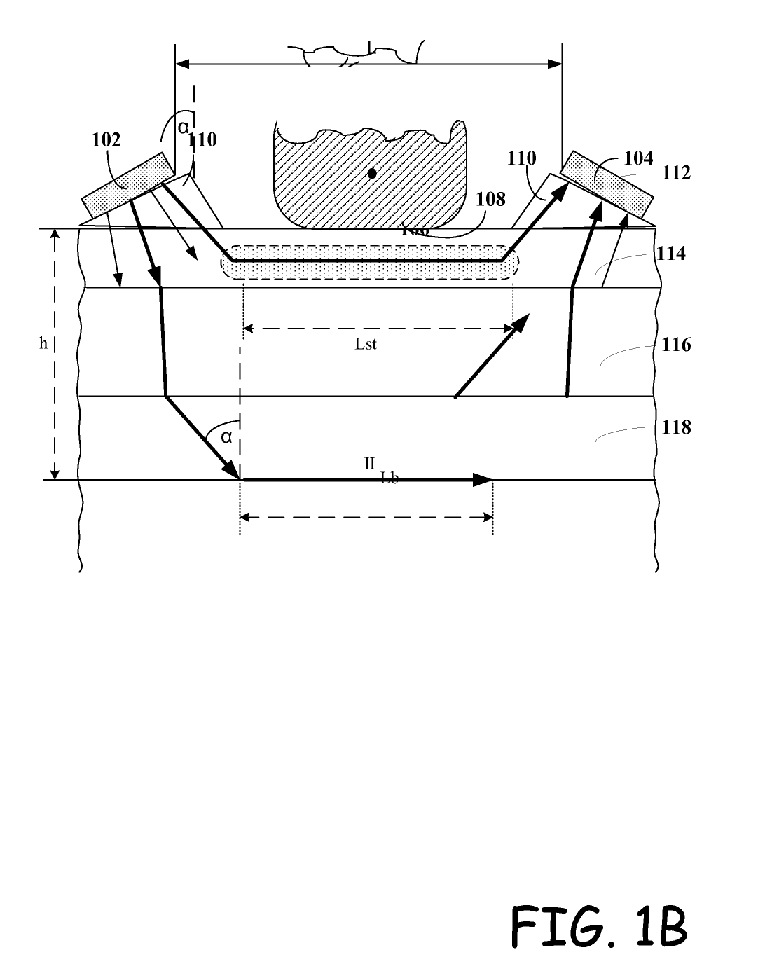

Reference is now made to FIG. 1A which is a simplified view of an exemplary embodiment of the current method and apparatus for precise ultrasound monitoring of treated skin temperature in real time employing a Brewster's angle of incidence. FIG. 1A illustrates a cross-sectional view of an exemplary embodiment of an aesthetic skin treatment device applicator 100. Applicator 100 includes an ultrasound transmitter 102 and an ultrasound receiver 104, each consisting of one or more piezoelectric elements (not shown). The piezoelectric elements may be constructed from one or more materials selected from a group consisting of ceramics, polymers and composites.

According to an exemplary embodiment of the current method and apparatus the transmitter and receiver are positioned at a predetermined distance from each other on opposing borders of an area of skin being treated 106 and at a predetermined angle relative to the surface of the skin. The angle between transmitter 102 and receiver 104 and the surface of the skin is maintained by a wedge 110 made of a sound index-matching material as known in the art. The index-matching material, such as a polymer (PVDF), liquid, cement (adhesive) or gel, has an index of refraction that closely approximates that of the medium adjacent to it, for example tissue layer 112, and is used to reduce reflection at the surface thereof. The distance between the transmitter and receiver is dependent on the thickness of the tissue at the area to be treated. The considerations determining the distance between the transmitter and receiver and the angle at which they are positioned relative to the surface of the skin will be explained in detail herein below.

Due to the physical-electrical nature of piezoelectric materials, it will be appreciated that transmitter 102 and receiver 104 may each function as a transceiver, emitting an ultrasound beam when excited by an electrical voltage received from a generator or converting a received ultrasound beam into an electrical voltage, amplified and delivered as a signal. The functionality of the transmitter 102 and receiver 104 may be dependent on the electrical circuitry configuration of apparatus 100 or controlled by a controller to determine the directionality of the transmitted ultrasound beams from transmitter 102 to receiver 104 or vice versa.

In the current exemplary embodiment applicator 100 may employ one or more sources of heating energy in a form of at least one of a group consisting of light, RF, ultrasound, electrolipophoresis, iontophoresis and microwaves and delivered to the tissue by heating surfaces. The current exemplary embodiment employs one or more RF electrodes heating surfaces 108 to heat skin 112 and/or subcutaneous fat 114. At proper treatment parameters, the applied energy heats area of skin 106, which includes skin and subcutaneous fat.

The elements of transmitter 102 and receiver 104 may be positioned in one or more predetermined configurations selected from a group consisting of two-dimensional and three-dimensional spatial configurations. Transmitter 102 and receiver 104 may also be positioned in a plurality of predetermined configurations in relation to heating surfaces 108. For example, View-A of FIG. 1A as illustrated in FIG. 1C, which is a plan view of aesthetic treatment device applicator 100 of FIG. 1A, illustrates transmitter 102 and receiver 104 positioned perpendicular to heat delivering surfaces 108 and on opposing borders of the tissue segment to be treated. In another exemplary embodiment of the current method and apparatus transmitter 102, receiver 104 and heat delivering surfaces 108 may be positioned on the same plane such as transmitter 102 and/or receiver 104 in-between two heating surfaces 108.

Reference is now made to FIG. 1B, which is a simplified cross section of another exemplary embodiment of the current method and apparatus for precise ultrasound monitoring of treated skin temperature in real time employing a Brewster's angle of incidence.

A transmitter 102 and a receiver 104 are positioned at a predetermined distance (L) from each other on opposing borders of an area 106 of skin tissue 112 being treated by energy delivered from heat delivery surface 108.

Transmitter 102 is operative to emit ultrasound beams, commonly in pulse form, at an angle relative to the surface of skin to be treated 112 so that a portion of the emitted beams impinges upon skin tissue 112 at a Brewster's angle of incidence, here indicated by the Greek letter (α). In light of the principle that ultrasound beams introduced into tissue at a Brewster's angle of incidence (α) propagate generally along the border between two mediums having two different sound refraction indexes, the beam emitted by transmitter 102 follows propagation path (I), which is generally parallel to the surface of skin tissue 112, through treated area 106 and along a distance (Lst). Skin tissue 112 emits the ultrasound beams at a Brewster's angle of incidence to be received by receiver 104. Receiver 104 converts the received ultrasound beams to signals communicated to a controller (Not shown).

During wave propagation inside the body, beams propagating through body tissues excite all particles to oscillate in all directions. Receiver 104 is, therefore, operative to receive most of the beams emitted by transmitter 102, at any distance there from and the signal value of the received beams depends on the transmitter-receiver distance.

Beams emitted into tissue layers 112, 114, 116 and 118 by transmitter 102 impinge on the surface of tissue 112 at a plurality of angles of incidence.

The fastest beams, i.e., the first to be received by receiver 104, are the beams travelled along the fastest transmitter 102-receiver 104 distance. The first beams to be received by receiver 104, i.e., those travelled along the fastest transmitter 102-receiver 104 distance, are those that have impinged on the surface of tissue layer 112 at a Brewster's angle of incidence and travelled along the surface of tissue 112 parallel thereto.

According to another exemplary embodiment of the current method and apparatus, the controller is operative to obtain from the ultrasound beam signals information regarding changes in propagation speed of the beams, which are indicative of the temperature changes in skin area 106 through which the beams have propagated. The controller then may compare the changes to a predetermined treatment protocol and determine the criticality of the changes, resulting in taking one or more actions based on the changes and criticality. Such actions may be, for example, one or more of the following: Record information relating to the changes and criticality in a database, display the information on a display, communicate the changes and criticality to a remote user, print the information on a printout, alert a user as to the changes based on their criticality and change the course of treatment based on the criticality. The controller is also operative to control each element in transmitter 102 and receiver 104 individually and determine the sequence of ultrasound beam pulse delivery.

According to yet another exemplary embodiment of the current method and apparatus, a portion of the beams emitted by transmitter 102 penetrate skin tissue 112 layer (L₁₁₂) and are refracted at the tissue layer borders due to differences in the sound refraction indexes between the various tissue layers. For example, beams travelling along propagation path (II) are emitted by transmitter 102 into tissue layer 112 and are refracted by the borders between adjacent tissue layers 112 (Skin) and 114 (L₁₁₄, Fat), refracted once again at the border between tissue layers 114 (Fat) and 116 (L₁₁₆, Muscle), and impinge upon a deeper tissue layer border, here being a border between layers 116 (Muscle) and 118 (Bone), at a Brewster's angle of incidence (α). In this case, the beam may then propagate along the border of deep tissue layers 116 and 118, following propagation path (II) along a distance (Lb) at the end of which it is deflected at a Brewster's angle of incidence, refracted once again along the propagation path towards the surface of skin 112 and emitted thereby.

Still referring to FIG. 1B and in accordance with another exemplary embodiment of the current method and apparatus determination of treated skin area 106 temperature, based on the ultrasound beam signals received from receiver 104 and communicated to the controller, may be obtained as follows:

The speed of sound wave propagation through various body tissues is well documented and may also be achieved empirically. It is also well documented that propagation speed of sound beams through tissue is temperature-dependent and is altered by any increase or decrease in tissue temperature. The approximated values of speed of sound in tissue at normal body temperature are as follows:

Skin: Velocity (V_{D}) ˜1700-1800 Meters per Second (m/s)

Fat: V ˜1460 m/s

Muscle: V 1580 m/s; and

Bone: Vb≧3000 m/s.

Soft tissue: Vst ˜1540 m/s. (average)

The propagation time of an ultrasound beam pulse along the path indicated by Roman numeral (I) may be calculated by employing the following formula:

$${τ}_{1} = \frac{L}{{V}_{D}}$$

Wherein (τ₁) is the time from ultrasound beam pulse emission by transmitter 102 to reception of the pulse by receiver 104, L is the distance between transmitter 102 and receiver 104 and (V_{D}) is the velocity of the beam along path (I).

Changes in treated tissue temperature may be determined in real time by comparison to known sound beam propagation speeds in non-heated various body tissues as brought hereinabove and sound beam propagation speed values at various tissue temperatures received empirically and recorded.

The propagation time of an ultrasound beam along the path indicated by Roman numeral (II) may be calculated employing the following formula:

$${τ}_{2} = \frac{2 {L}_{S t}}{{V}_{S t}} + \frac{{L}_{B}}{{V}_{B}} = \frac{2 {L}_{S t}}{{V}_{S t}} + \frac{L - 2 h tan α}{{V}_{B}}$$

Wherein (τ₂) is the time from emission of the pulse by transmitter 102 to reception of the pulse by receiver 104, (Lst) is the distance of beam propagation trough layers of soft tissue (Lst=L₁₁₂+L₁₁₄+L₁₁₆), (L_{B}) is the distance of beam propagation along the bone surface layer, (L) is the distance between transmitter 102 and receiver 104, (h) is the thickness of the tissue layers measured from the border between bone surface layer 118 and skin layer 112, (Vst) is the velocity of the beam propagation through soft tissue, (V_{B}) is the velocity in the bone 118, (α) is Brewster's angle and wherein

$$Sin α = \frac{{V}_{St}}{{V}_{B}} .$$

It will be appreciated from the above expression that the time ((τ₂) of propagation of a sound beam travelling along path (II) depends on the thickness of the tissue layers in treated area 106, between the surface of skin tissue 112 and the muscle 116-bone 118 border. Sound propagation speed along bone (V_{B}>3000 m/s) is more than twice the propagation speed of sound within soft tissue, hence in cases where tissue thickness (h) is small as compared with L, a sound beam travelling along path (II) and travelling distance L_{B} at a speed (Vb) may be received by receiver 104 before or at the same time as a sound beam travelling along path (I) and travelling distance L at a much slower speed (V_{D}). This may result in the sound beam travelling along path (II) masking the signal received from the sound beam travelling along path (I). This places the condition that the sound beam travelling along path (I) is received before the sound beam travelling along path (II) or that (τ₁)<(τ₂). This may be achieved by using the following expression:

$$\frac{L}{{V}_{D}} \leq \frac{2 h \sqrt{1 + {tan}^{2} α}}{{V}_{S t}} + \frac{L - 2 h tan α}{{V}_{B}}$$

### Therefore:

$$h \geq \frac{L ( \frac{1}{{V}_{D}} - \frac{1}{{V}_{B}} )}{2 ( \frac{\sqrt{1 + {tan}^{2} α}}{{V}_{St}} - \frac{tan α}{{V}_{B}} )}$$

and the distance (L) between transmitter 102 and receiver 104 may be determined according to the thickness (h).

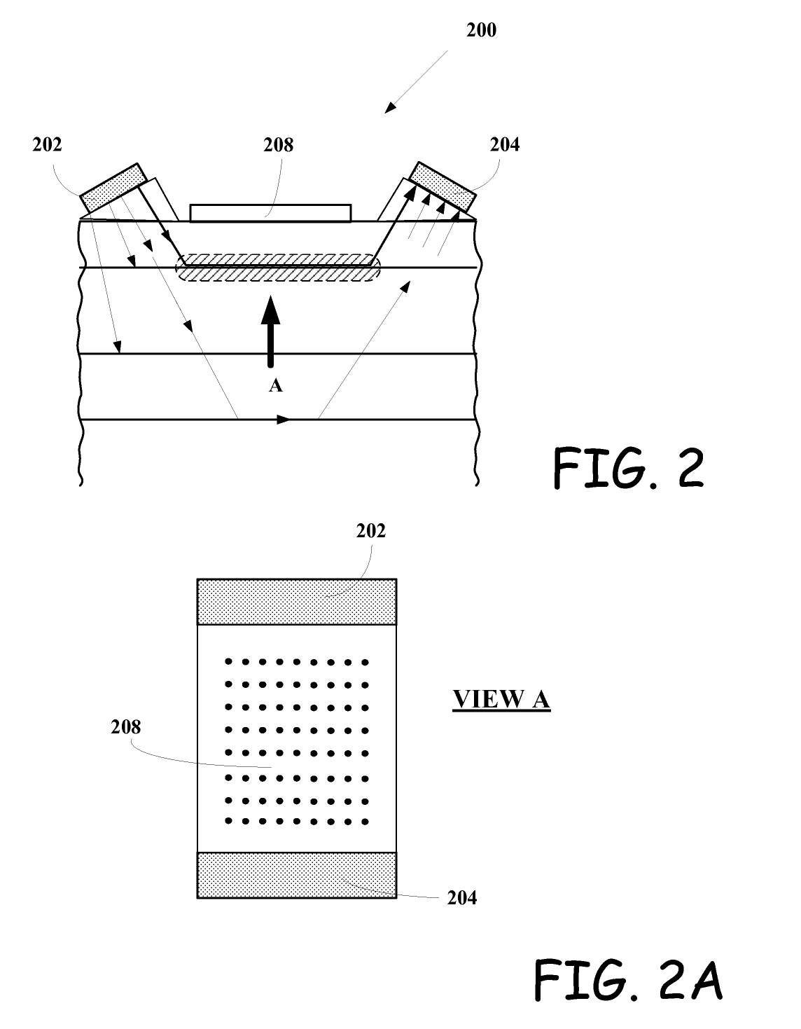

Reference is now made to FIG. 2A, which is a cross-sectional view of another exemplary embodiment of an aesthetic skin treatment device applicator 200 in which the heating energy delivery surface 208 is an RF matrix and is defined by opposing ultrasound transmitter 202 and ultrasound receiver 204. View A, as illustrated in FIG. 2B, is a plan view aesthetic treatment device applicator 200 of FIG. 2A.

It will be appreciated by persons skilled in the art that the present method and apparatus are not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

What is claimed is:

1. An apparatus for real time precision ultrasound monitoring of aesthetic treatments applied to body tissue, the apparatus comprising:
a housing including:
a transmitter operative to emit ultrasound beams into said tissue at a predetermined angle of incidence;
a receiver located at a predetermined distance from said transmitter and operative to receive ultrasound beams emitted by said transmitter, propagated through said tissue substantially in parallel to the surface thereof and emitted thereby; and
a controller operative to
obtain from said received ultrasound beams information regarding changes in propagation speed of said beams through said tissue; and
analyze said information to determine changes in at least one treatment effect on said tissue.

2. The apparatus according to claim 1, and wherein said angle of incidence is a Brewster's angle of incidence.

3. The apparatus according to claim 1, and wherein said treatment effect is tissue temperature.

4. The apparatus according to claim 1, and wherein said tissue is body tissue layers and wherein said receiver is also operative to receive transmitted ultrasound beams propagated generally along an inter-layer border between said layers and emitted thereby.

5. The apparatus according to claim 1, and wherein said receiver is also operative to receive ultrasound beams emitted by said tissue at said angle of incidence.

6. The apparatus according to claim 1, and wherein said transmitter and receiver each also comprising at least one piezoelectric element constructed from at least one piezoelectric material selected from a group consisting of ceramics, polymers and composites.

7. The apparatus according to claim 1, and wherein said transmitter and receiver each also comprising at least two piezoelectric elements positioned in at least one predetermined configuration selected from a group consisting of two-dimensional and three-dimensional spatial configurations.

8. The apparatus according to claim 7, and wherein said elements are constructed from at least one piezoelectric material selected from a group consisting of ceramics, polymers and composites.

9. The apparatus according to claim 7, and wherein said controller is also operative to control each of said elements individually.

10. The apparatus according to claim 1, and wherein said predetermined distance is dependent on the thickness of said tissue in the area located between said transmitter and said receiver.

11. The apparatus according to claim 1, and wherein said predetermined distance is adjustable.

12. The apparatus according to claim 7, and wherein said controller is also operative to control each of said elements individually depending on the thickness of said tissue in the area located between said transmitter and said receiver.

13. The apparatus according to claim 1, and wherein said transmitter is also operative to emit at least two ultrasound beams at a predetermined sequence.

14. The apparatus according to claim 1, and wherein said apparatus also comprises at least one generator operative to excite oscillation of said transmitter and bring about emission of ultrasound beams.

15. The apparatus according to claim 1, and wherein said beams are ultrasound pulse beams.

16. The apparatus according to claim 1, and wherein said apparatus also comprises at least one amplifier operative to amplify signals received by said receiver.

17. The apparatus according to claim 1, and wherein said controller is also operative in real time to:
compare said changes to a predetermined treatment protocol and determine the criticality thereof; and
take at least one action based on said changes and criticality.

18. The apparatus according to claim 17, and wherein said action comprises at least one of the following:
record information relating to said changes and criticality in a database;
display said information on a display;
communicate said changes and criticality to a remote user;
print said information on a printout;
alert a user as to said changes based on said criticality; and
change the course of treatment based on said criticality.

19. The apparatus according to claim 1, and wherein said treatments comprise at least one aesthetic skin tissue treatment selected from a group consisting of sub-dermal fat cells breakdown, lessening of the amount of sub-dermal fat, tightening of loose skin, tightening and firming of body surfaces, reduction of wrinkles in the skin and collagen remodeling.

20. An apparatus for real time precision ultrasound monitoring of aesthetic treatments applied to body tissue, the apparatus comprising:
a housing including:
a transmitter operative to emit ultrasound beams into said tissue at a Brewster's angle of incidence;
a receiver located at a predetermined distance from said transmitter and operative to receive ultrasound beams emitted by said transmitter, propagated through said tissue, substantially in parallel to the surface thereof, and emitted thereby; and
a controller operative to
obtain from said received ultrasound beams information regarding changes in propagation speed of said beams through said tissue; and
analyze said information to determine changes in at least one treatment effect on said tissue.

21. An apparatus for real time precision ultrasound monitoring of aesthetic treatments applied to body tissue layers, the apparatus comprising:
a housing including:
a transmitter operative to emit ultrasound beams into said layers at a Brewster's angle of incidence;
a receiver located at a predetermined distance from said transmitter and operative to receive ultrasound beams emitted by said transmitter, propagated generally along an inter-layer border between said layers and emitted thereby; and
a controller operative to
obtain from said received ultrasound beams information regarding changes in propagation speed of said beams through said tissue; and
analyze said information to determine changes in at least one treatment effect on said tissue.

22. The apparatus according to claim 1, and wherein said apparatus also comprises at least one heating energy delivery surface supplied by a source of heating energy.

23. The apparatus according to claim 22, and wherein said heating energy is in a form of at least one of a group consisting of light, RF, ultrasound, electrolipophoresis, iontophoresis and microwaves.

24. The apparatus according to claim 22, and wherein said transmitter and receiver each also comprising at least one piezoelectric element positioned generally perpendicular to said heating energy delivery surface.

25. The apparatus according to claim 22, and wherein said transmitter and receiver each also comprising at least one piezoelectric element positioned on one plane with said heating energy delivery surface.

26. The apparatus according to claim 22, and wherein said energy delivery surface is an RF matrix and wherein said transmitter and receiver are positioned each at opposite ends of said RF matrix.

27. The apparatus according to any of the preceding claims, and wherein said tissue also comprises bone.

28. An apparatus for real time precision ultrasound monitoring of aesthetic treatments applied to body tissue, the apparatus comprising:
a housing including:
at least one pair of transceivers each consisting of
a first transceiver operative to emit ultrasound beams into said tissue at a predetermined angle of incidence; and
a second transceiver operative to receive ultrasound beams emitted by said first transceiver, propagated through said tissue substantially in parallel to the surface thereof and emitted thereby; and
a controller operative to
obtain from said received ultrasound beams information regarding changes in propagation speed of said beams through said tissue; and
analyze said information to determine changes in at least one treatment effect on said tissue.

29. The apparatus according to claim 28, and wherein also said second transceiver is operative to emit ultrasound beams into said tissue at a predetermined angle of incidence; and
said first transceiver is operative to receive ultrasound beams emitted by said first transceiver, propagated through said tissue substantially in parallel to the surface thereof and emitted thereby.

30. A method for real time precision ultrasound monitoring of aesthetic treatments applied to body tissue, the method comprising:
emitting at a predetermined angle of incidence ultrasound beams into tissue being treated;
receiving transmitted ultrasound beams propagated through said tissue, substantially in parallel to the surface thereof, and emitted thereby;
obtaining from said received ultrasound beams information regarding changes in propagation speed of said beams through said tissue; and
analyzing said information to determine changes in at least one treatment effect on said tissue.

31. The method according to claim 30, and wherein said angle of incidence is a Brewster's angle of incidence.

32. The method according to claim 30, and wherein also comprising receiving ultrasound beams emitted by said tissue at said angle of incidence.

33. The method according to claim 30, and wherein also comprising transmitting at least two ultrasound beams in a predetermined sequence.

34. The method according to claim 30, and wherein said beams are ultrasound pulse beams.

35. The method according to claim 30, and wherein also comprising amplifying signals of said received ultrasound beams.

36. The method according to claim 30, and wherein also comprising in real time
comparing said changes to a predetermined treatment protocol and determining the criticality thereof; and
taking at least one action based on said changes and criticality.

37. The method according to claim 36, and wherein said action comprises at least one of the following:
recording information relating to said changes and criticality in a database;
communicating said changes and criticality to a remote user;
displaying said information on a display;
printing said information on a printout;
alerting a user as to said changes based on said criticality; and
changing the course of treatment based on said criticality.

38. The method according to claim 30, and wherein said aesthetic treatments also comprise
breaking down sub-dermal fat cells, lessening the amount of sub-dermal fat, tightening loose skin, tightening and firming body surface, reducing wrinkles in the skin and remodeling collagen.

39. A method for real time precision ultrasound monitoring of aesthetic treatments applied to body tissue layers, the method comprising:
emitting ultrasound beams into said tissue at a Brewster's angle of incidence;
receiving ultrasound beams, propagated generally along an inter-layer border between said layers and emitted thereby;
obtaining from said received ultrasound beams information regarding changes in propagation speed of said beams through said tissue; and
analyzing said information to determine changes in at least one treatment effect on said tissue.

40. The method according to claim 30, and wherein also comprising changing the course of treatment based on said changes in said treatment effect.

41. The method according to claim 30, and wherein also comprising applying heating energy to said tissue.

42. The method according to claim 41, and wherein said heating energy is in a form of at least one of a group consisting of light, RF, ultrasound, electrolipophoresis, iontophoresis and microwaves.

43. The method according to claim 41, and wherein also comprising applying said heating energy in a direction generally perpendicular to the direction of said transmitted ultrasound beams.

44. The method according to claim 41, and wherein also comprising applying said heating energy in a direction generally parallel to the direction of said transmitted ultrasound beams.

45. The method according to any of the preceding claims 30-44, and wherein said tissue also comprises bone.
